(19) United States
(12) Patent Application Publication
(10) Pub. No.: US 2025/0352704 A1
(43) Pub. Date: Nov. 20, 2025

(54) BREAST PUMP BRA

(71) Applicant: Shuqin WU, Shenzhen (CN)

(72) Inventor: Shuqin WU, Shenzhen (CN)

(73) Assignee: Shenzhen Aobeite Technology Service Co., Ltd., Shenzhen (CN)

(21) Appl. No.: 18/737,112

(22) Filed: Jun. 7, 2024

(30) Foreign Application Priority Data
May 14, 2024 (CN) 202421049639.4

(51) Int. Cl. A61M 1/06

(52) U.S. Cl. CPC A61M 1/067; A61M 1/064

(57) ABSTRACT

Disclosed in the prevent invention is a breast pump bra, including a first fabric layer and a second fabric layer, where the first fabric layer is provided with first notches from cup center areas covering breasts to peripheral side areas, the second fabric layer passes through each of the first notches and overlaps the first fabric layer in a layered manner, cross bearing openings are formed at overlapping portions of first force-bearing edges of the first fabric layer and second force-bearing edges of the second fabric layer; and under the action of an external force, each of the cross bearing openings is capable of enlarging an opening area thereof along each of the cup center areas to each of the cup peripheral side areas.

### CROSS REFERENCE TO RELATED APPLICATIONS

This application claims priority benefits to Chinese Patent Application No. 202421049639.4, filed on May 14, 2024, the contents of which are incorporated herein by reference.

### TECHNICAL FIELD

The present invention relates to the technical field of underwear, and in particular, to a breast pump bra.

### BACKGROUND

Breast pumps are mainly used for helping lactating women to extract milk. Breast pump bras generally use an opening designed on the bra that can support a flange port of the breast pump so as to release two hands. However, the design of existing breast pump bras often have some problems, which affect the user's experience. Firstly, the design of the opening has a poor adaptability, and a product cannot be suitable for breast pumps having different sizes, and when the size is not suitable, the flange port of the breast pump is likely to be poorly adhered to the breast, thereby affecting the suction process; secondly, the support of the opening is poor, the structure of the opening of some products is relatively weak, and when a user moves her body during use, the support of the breast pump will be unstable. Thirdly, in order to improve the support of the opening to the breast pump, the structures of the bras are limited after the opening is designed for some products, causing it uncomfortable for a user to wear, for example, for lactating women, lactating women may experience discomfort when wearing a bra after their breast size increases due to milk engorgement.

### SUMMARY

The object of the present invention is to disclose a breast pump bra, so as to solve the problem of applicability of breast pumps having different sizes, and to provide a more stable supporting effect, so that a user can use the breast pump with more confidence.

In order to achieve the above object, the present invention discloses the following technical solution.

A breast pump bra, comprising a first fabric layer and a second fabric layer, wherein the first fabric layer is provided with first notches from cup center areas covering breasts to cup peripheral side areas, the second fabric layer passes through each of the first notches and overlaps the first fabric layer in a layered manner, cross bearing openings are formed at overlapping portions of first force-bearing edges of the first fabric layer and second force-bearing edges of the second fabric layer, under the action of an external force, each of the cross bearing openings is capable of enlarging an opening area thereof along each of the cup center areas to each of the cup peripheral side areas so as to place a flange port of a breast pump, and each of the cross bearing openings is capable of surrounding and wrapping the outer diameter of a suction opening of the flange port of the breast pump by means of tension of fabrics of the first fabric layer and the second fabric layer.

Further, each of the first force-bearing edges of the first fabric layer and each of the second force-bearing edges of the second fabric layer are both concave, each of the first force-bearing edges is opposite to each of the second bearing edges in a recess direction at each of the cross bearing openings, and two tension directions for reducing the opening area of each of the cross bearing openings formed at the cross bearing openings by the second fabric layer and the first fabric layer are opposite.

Further, each of the first force-bearing edges of the first fabric layer is concave downwards, each of the second force-bearing edges of the second fabric layer is concave upwards, and two tension directions for reducing the opening area of each of the cross bearing openings formed at the cross bearing openings by the second fabric layer and the first fabric layer are opposite.

Further, the first notches are respectively formed by extending from the two cup center areas to a symmetric center of two cups in the cup peripheral side areas, so that the first force-bearing edge of each of the first notches forms a closed ring shape; and when an external force is applied to enlarge each of the first notches, the first force-bearing edge of each of the first notches has tension limiting the area of each of the first notches to be enlarged.

Further, each of the first notches extends from each of the cup center areas covering the breasts to an edge of each of the cup peripheral side areas so as to form a first slit, and each of the first slits enables the second fabric layer to conveniently pass through each of the first notches.

Further, the first fabric layer comprises two first notches, and the two first notches respectively extend from the cup center areas edges of the cup peripheral side areas.

Further, the second fabric layer comprises two second notches, and the second force-bearing edges of the two second notches respectively extend from the two cup center areas to the edges of the cup peripheral side areas.

Further, each of the second notches forms a second slit at the edge of each of the cup peripheral side areas.

Further, two first notches having a downward concave shape are provided, and the two first notches extend from the cup center areas covering the breasts to the corresponding cup peripheral side areas so as to form first slits, the two first slits enable the first fabric layer to extend downwards from the symmetrical center to a center front portion so as to form a first lower portion having a downward tension force, and the second fabric layer passes through the two first notches respectively and overlaps the first fabric layer in a layered manner so as to form the bearing openings.

Further, each of the second notches having a downward concave shape extends from each of the cup center areas covering the breasts to each of the cup peripheral side areas in different directions to form a second slit, and the two slits enable the second fabric layer to extend downwards from the symmetry center to a center front portion so as to form a second lower portion having a downward tension force.

Further, the nursing bra further comprises a third fabric layer, wherein the third fabric layer covers an outer side of the first fabric layer or the second fabric layer, and covers the bearing openings.

Further, the nursing bra further comprises side panels and an underband, wherein the first fabric layer, the second fabric layer and the third fabric layer are fixedly connected to the side panels and the underband respectively.

It can be seen by analysis that the present invention discloses a breast pump bra. By setting the first notches on the first fabric layer, and enabling side parts at two sides of the second fabric layer to respectively pass through the two first notches and be overlap the first fabric layer, the bearing openings capable of surrounding and wrapping a flange port of a breast pump is formed between a force-bearing edge of each of the first notches and a force-bearing edge of the second fabric layer, and the breast pump has a more stable bearing. In the present invention, the bearing openings formed by the cross-stacking of two fabrics can be suitable for breast pumps having various sizes. In addition, the bearing openings formed by cross-stacking of two fabrics can reduce the limitation of the bra structure, making it more comfortable to wear compared to a breast pump bra having bearing openings formed by single piece overlapping. The main function of the bearing opening is to stably wrap and support the flange port part of the breast pump, so as to ensure that the flange port can be firmly located at a correct position during use, and maintain good contact with the mother's breast, thereby improving the breast suction efficiency and comfort. In addition, the double-layer fabric design also helps to improve the durability and comfort of the product, and provides a better user experience.

### BRIEF DESCRIPTION OF DRAWINGS

The accompanying drawings, which form a part of the present application, are used to disclose a further understanding of the present invention. The schematic embodiments of the present invention and the description thereof are used to explain the present invention, and do not form improper limits to the present invention. In the drawings:

FIG. 1 is a schematic view of the structure of a first fabric, a second fabric, and their overlapping front and back of the breast pump bra according to a first embodiment;

FIG. 2 is a schematic view of the structure of a first fabric, a second fabric, and their overlapping front and back of the breast pump bra according to a second embodiment;

FIG. 3 is a schematic view of the structure of a first fabric, a second fabric, and their overlapping front and back of the breast pump bra according to a third embodiment;

FIG. 4 is a schematic view of the structure of a first fabric, a second fabric, and their overlapping front and back of the breast pump bra according to a fourth embodiment;

FIG. 5 is a schematic view of the structure of a first fabric, a second fabric, and their overlapping front and back of the breast pump bra according to a fifth embodiment;

FIG. 6 is a schematic view of the structure of a first fabric, a second fabric, and their overlapping front and back of the breast pump bra according to a sixth embodiment;

FIG. 7 is a schematic view of the structure of a first fabric, a second fabric, and their overlapping front and back of the breast pump bra according to a seventh embodiment;

FIG. 8 is a schematic view of the structure of a first fabric, a second fabric, and their overlapping front and back of the breast pump bra according to an eighth embodiment;

FIG. 9 is a schematic view of the structure of a first fabric, a second fabric, and their overlapping front and back of the breast pump bra according to a ninth embodiment;

FIG. 10 is a schematic view of the structure of a first fabric, a second fabric, and their overlapping front and back of the breast pump bra according to a tenth embodiment;

FIG. 11 is a schematic view of the structure of a first fabric, a second fabric, and their overlapping front and back of the breast pump bra according to an eleventh embodiment;

FIG. 12 is a schematic view of the structure of a first fabric, a second fabric, and their overlapping front and back of the breast pump bra according to a twelfth embodiment;

FIG. 13 is a schematic view of the structure of a first fabric, a second fabric, and their overlapping front and back of the breast pump bra according to a thirteenth embodiment;

FIG. 14 is a schematic view of the front and back of a breast pump bra according to embodiments after a first fabric layer and a second fabric layer are overlapped and are combined with a third fabric layer;

### DESCRIPTION OF REFERENCE NUMERALS

1—first fabric layer; 10—first force-bearing edge; 11—first notch; 12—first slit;
2—second fabric layer; 20—second force-bearing edge; 21—second notch; 22—second slit;
30—cup center area; 31—cup peripheral side area; 32—symmetric center; 33—center front portion; 34—side panel; 35—underband; 36—cup strap; 37—cup buckle;
3—third fabric layer; 38—side notches; and
100—cross bearing opening;

### DESCRIPTION OF EMBODIMENTS

The present invention will be described below with reference to the drawings and embodiments in detail. The respective examples are disclosed by way of explanation of the present invention without limiting the present invention. Indeed, it will be apparent to a person skilled in the art that modifications and variations can be made in the present invention without departing from the scope or spirit of the invention. For example, features illustrated or described as being part of one embodiment may be used in another embodiment to yield yet another embodiment. Therefore, it is expected that the present invention includes such modifications and variations within the scope of the accompanying claims and their equivalent.

In the description of the present invention, orientation or position relationships indicated by terms such as “longitudinal”, “lateral”, “up”, “down”, “front”, “rear”, “left”, “right”, “vertical”, “horizontal”, “top”, and “bottom” are based on orientation or position relationships shown in the accompanying drawings, which are only used to facilitate description of the present invention rather than requiring that the present invention must be constructed and operated in a specific orientation, and therefore cannot be construed as a limitation on the present invention. The terms “connecting”, “connected” and “provided” used in the present invention should be understood broadly, for example, may be fixedly connected, and may also be detachably connected; may also be direct connections or indirect connections via intervening components; may also be wired connections or radio connections; and may also be wireless communication signal connections. A person of ordinary skill in the art would have been able to understand the specific meaning of the described terms according to specific situations.

One or more examples of the present invention are illustrated in the accompanying drawings. The detailed description uses numerals and letter markings to refer to features in the drawings. The similar or analogous markings in the drawings and descriptions have been used to refer to similar or analogous parts of the present invention. As used herein, the terms “first”, “second”, and “third”, etc., are used interchangeably to distinguish one member from another, and are not intended to represent the location or importance of the individual members.

In the breast pump bra disclosed in the present invention, cup center areas 30 are areas covering human nipples and areolas; the cup peripheral side areas 31 are areas covering the breasts except the human nipples and the areolas; the first notches 11 and the second notches 21 are respectively notches or reserve openings of the fabrics, and the first force-bearing edges 10 correspond to the edges of the notches of the first fabric layer 1 on which the first notches 11 are located; the second force-bearing edges 20 correspond to the edges of the notches of the second fabric layer on which the second notches 21 are located. The material of the first force-bearing edges 10 and the second force-bearing edges 20 can be fabric itself, and may also be an elastic fabric, an elastic string or an expanding yarn, or the like, so as to perform edge locking and reinforcement to form an edge of an elastic notch.

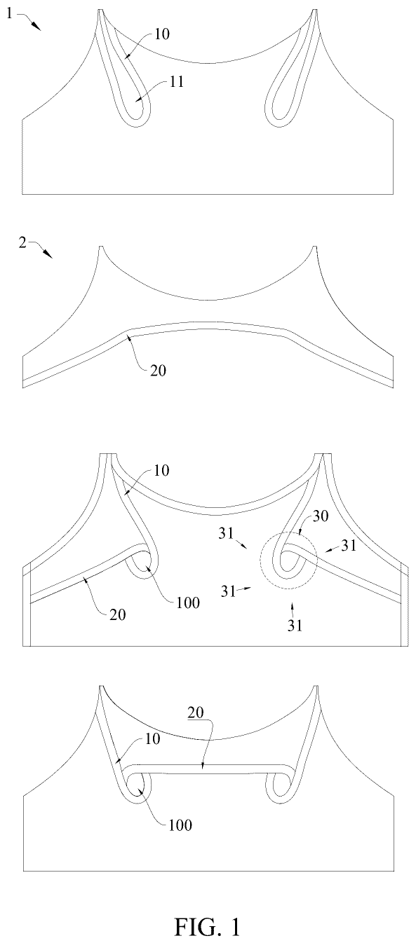

As shown in FIG. 1, a breast pump bra disclosed in a first embodiment of the present invention comprises a first fabric layer 1 and a second fabric layer 2, in which the first fabric layer 1 is provided with first notches 11 in two cups respectively from cup center areas 30 covering breasts to cup peripheral side areas 31 thereof, each of the first force-bearing edges 10 of each of the first notches 11 is concave upwards, and the overall shape of each of the second force-bearing edges 20 of the second fabric layer 2 is concave downwards.

The second fabric layer 2 passes through the two first notches 11 and overlaps the first fabric layer 1 in a layered manner, two cross bearing openings 100 are formed at overlapping portions of first force-bearing edges 10 of the first fabric layer 1 and second force-bearing edges 20 of the second fabric layer 2, under the action of an external force, the two cross bearing openings 100 are capable of enlarging opening areas thereof along the cup center areas 30 to the cup peripheral side areas 31, after a flange port of a breast pump is placed, the cross bearing openings 100 are capable of surrounding and wrapping the outer diameter of a suction opening of the flange port of the breast pump by means of the tension of the first fabric layer 1 and the second fabric layer 2.

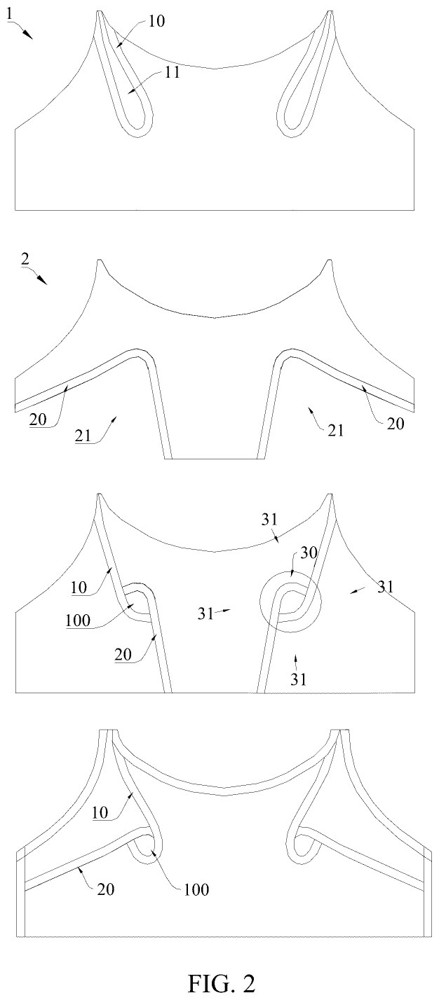

As shown in FIG. 2, a breast pump bra disclosed in a second embodiment of the present invention comprises a first fabric layer 1 and a second fabric layer 2, in which the first fabric layer 1 is provided with first notches 11 in two cups respectively from cup center areas 30 covering breasts to cup peripheral side areas 31 thereof, each of the first force-bearing edges 10 of each of the first notches 11 is concave upwards; two second notches 21 are cut at the second fabric layer 2 in two cups respectively from cup center areas 30 covering breasts to the edges of cup peripheral side areas 31 thereof, the second fabric layer 2 passes through each of the first notches 11 and overlaps with the first fabric layer 1 in a layered manner, two cross bearing openings 100 are formed at overlapping portions of second force-bearing edges 20 of the second notches 21 and first force-bearing edges 10 of the first notches 11, under the action of an external force applied, the two cross bearing openings 100 are capable of enlarging opening areas thereof along the cup center areas 30 to the cup peripheral side areas 31; after a flange port of a breast pump is placed, the cross bearing openings 100 are capable of surrounding and wrapping the outer diameter of a suction opening of the flange port of the breast pump by means of the tension of the first fabric layer 1 and the second fabric layer 2.

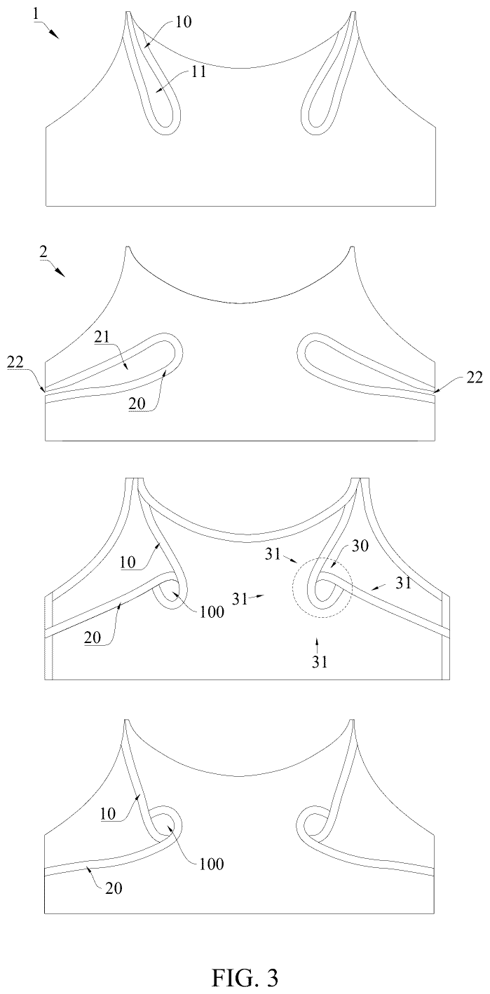

As shown in FIG. 3, a breast pump bra disclosed in a third embodiment of the present invention comprises a first fabric layer 1 and a second fabric layer 2, in which the first fabric layer 1 is provided with first notches 11 in two cups respectively from cup center areas 30 covering breasts to cup peripheral side areas 31 thereof, each of the first force-bearing edges 10 of each of the first notches 11 is concave upwards; two second notches 21 are cut at the second fabric layer 2 in two cups respectively from cup center areas 30 covering breasts to the edges of cup peripheral side areas 31 thereof, each of the second notches 21 forms a second slit 22 extending to each of the cup peripheral side areas 31, the second fabric layer 2 passes through each of the first notches 11 and overlaps with the first fabric layer 1 in a layered manner, two cross bearing openings 100 are formed at overlapping portions of second force-bearing edges 20 of the second notches 21 and first force-bearing edges 10 of the first notches 11, under the action of an external force applied, the two cross bearing openings 100 are capable of enlarging opening areas thereof along the cup center areas 30 to the cup peripheral side areas 31; after a flange port of a breast pump is placed, the cross bearing openings 100 are capable of surrounding and wrapping the outer diameter of a suction opening of the flange port of the breast pump by means of the tension of the first fabric layer 1 and the second fabric layer 2.

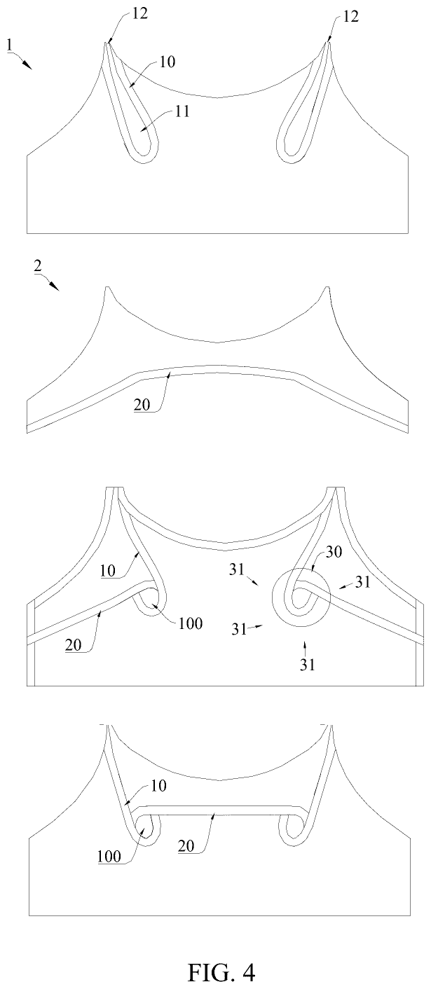

As shown in FIG. 4, a breast pump bra disclosed in a fourth embodiment of the present invention comprises a first fabric layer 1 and a second fabric layer 2, in which the first fabric layer 1 is provided with first notches 11 in two cups respectively from cup center areas 30 covering breasts to cup peripheral side areas 31 thereof, the first force-bearing edges 10 of the two first notches 11 are concave upwards, each of the first notches 11 extends from each of the cup center areas 30 covering the breasts to an edge of each of the cup peripheral side areas 31 so as to form a first slit 12, the slits 12 enable the second fabric layer 2 to conveniently pass through the first notches 11.

The overall shape of each of the second force-bearing edges 20 of the second fabric layer 2 is concave downwards, the second fabric layer 2 passes through the two first notches 11 and overlaps the first fabric layer 1 in a layered manner, two cross bearing openings 100 are formed at overlapping portions of first force-bearing edges 10 of the first fabric layer 1 and second force-bearing edges 20 of the second fabric layer 2, under the action of an external force applied, the two cross bearing openings 100 are capable of enlarging opening areas thereof along the cup center areas 30 to the cup peripheral side areas 31, after a flange port of a breast pump is placed, the cross bearing openings 100 are capable of surrounding and wrapping the outer diameter of a suction opening of the flange port of the breast pump by means of the tension of the first fabric layer 1 and the second fabric layer 2.

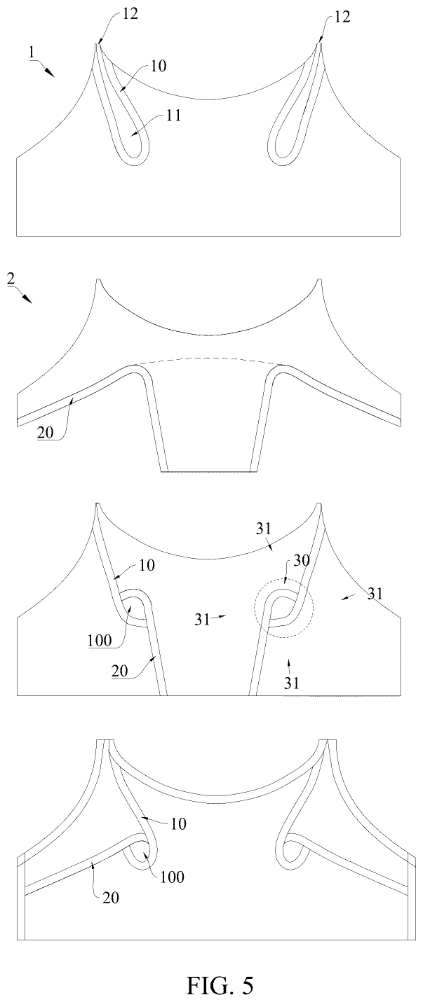

As shown in FIG. 5, a breast pump bra disclosed in a fifth embodiment of the present invention comprises a first fabric layer 1 and a second fabric layer 2, in which the first fabric layer 1 is provided with first notches 11 in two cups respectively from cup center areas 30 covering breasts to cup peripheral side areas 31 thereof, each of the first force-bearing edges 10 of each of the first notches 11 is concave downwards; and each of the first notches 11 extends from each of the cup center areas 30 covering the breasts to an edge of each of the cup peripheral side areas 31 so as to form a first slit 12, and the slits 12 enable the second fabric layer 2 to conveniently pass through the first notches 11.

Two second notches 21 are cut at the second fabric layer 2 in two cups respectively from cup center areas 30 covering breasts to the edges of cup peripheral side areas 31 thereof, the second fabric layer 2 passes through each of the first notches 11 and overlaps with the first fabric layer 1 in a layered manner, two cross bearing openings 100 are formed at overlapping portions of second force-bearing edges 20 of the second notches 21 and first force-bearing edges 10 of the first notches 11, under the action of an external force applied, the two cross bearing openings 100 are capable of enlarging opening areas thereof along the cup center areas 30 to the cup peripheral side areas 31; after a flange port of a breast pump is placed, the cross bearing openings 100 are capable of surrounding and wrapping the outer diameter of a suction opening of the flange port of the breast pump by means of the tension of the first fabric layer 1 and the second fabric layer 2.

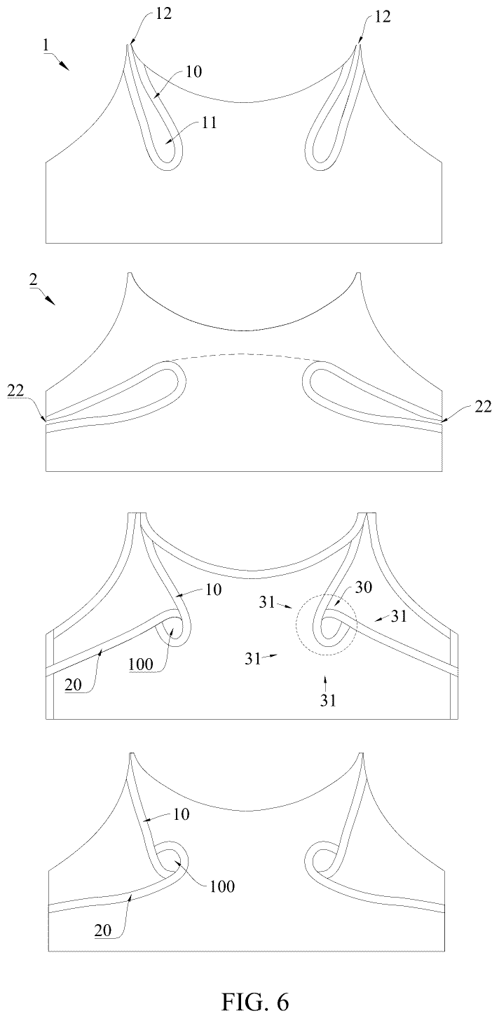

As shown in FIG. 6, a breast pump bra disclosed in a sixth embodiment of the present invention comprises a first fabric layer 1 and a second fabric layer 2, in which the first fabric layer 1 is provided with first notches 11 in two cups respectively from cup center areas 30 covering breasts to cup peripheral side areas 31 thereof, the first force-bearing edges 10 of each of the first notches 11 are concave downwards, each of the first notches 11 extends from each of the cup center areas 30 covering the breasts to an edge of each of the cup peripheral side areas 31 so as to form a first slit 12, the slits 12 enable the second fabric layer 2 to conveniently pass through the first notches 11.

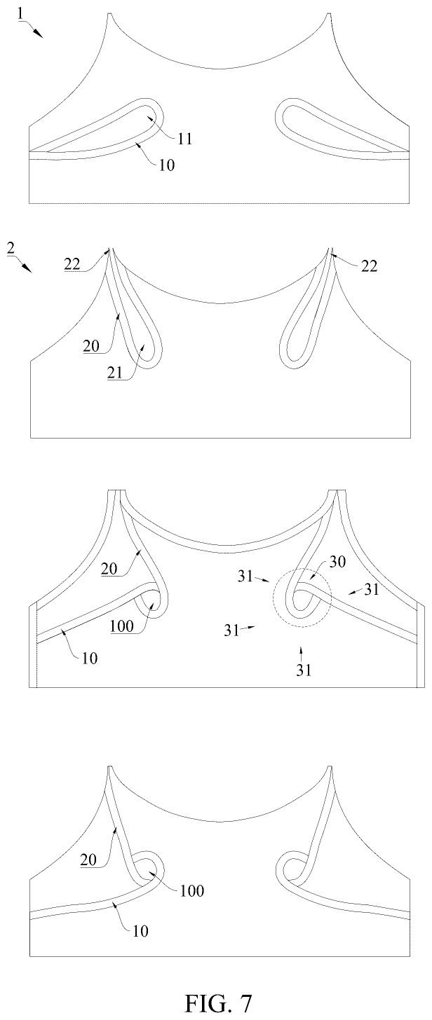

Two second notches 21 are cut at the second fabric layer 2 in two cups respectively from cup center areas 30 covering breasts to the edges of cup peripheral side areas 31 thereof, each of the second notches 21 forms a second slit 22 extending to each of the cup peripheral side areas 31, the second fabric layer 2 passes through each of the first notches 11 and overlaps with the first fabric layer 1 in a layered manner, two cross bearing openings 100 are formed at overlapping portions of second force-bearing edges 20 of the second notches 21 and first force-bearing edges 10 of the two first notches 11, under the action of an external force applied, the two cross bearing openings 100 are capable of enlarging opening areas thereof along the cup center areas 30 to the cup peripheral side areas 31; after a flange port of a breast pump is placed, the cross bearing openings 100 are capable of surrounding and wrapping the outer diameter of a suction opening of the flange port of the breast pump by means of the tension of the first fabric layer 1 and the second fabric layer 2. As shown in FIG. 7, a breast pump bra disclosed in a seventh embodiment of the present invention comprises a first fabric layer 1 and a second fabric layer 2, in which the first fabric layer 1 is provided with first notches 11 in two cups respectively from cup center areas 30 covering breasts to cup peripheral side areas 31 thereof, each of the first force-bearing edges 10 of each of the first notches 11 is closed downward concave.

Two second notches 21 having an upward concave shape are cut at the second fabric layer 2 in two cups respectively from cup center areas 30 covering breasts to the edges of cup peripheral side areas 31 thereof, each of the second notches 21 extends to each of the cup peripheral side areas 31 so as to form a second slit 22, the second fabric layer 2 passes through each of the first notches 11 and overlaps with the first fabric layer 1 in a layered manner, two cross bearing openings 100 are formed at overlapping portions of second force-bearing edges 20 of the two second notches 21 and the first force-bearing edges 10 of the two first notches 11, under the action of an external force, the two cross bearing openings 100 are capable of enlarging opening areas thereof along the cup center areas 30 to the cup peripheral side areas 31; after a flange port of a breast pump is placed, the cross bearing openings 100 are capable of surrounding and wrapping the outer diameter of a suction opening of the flange port of the breast pump by means of the tension of the first fabric layer 1 and the second fabric layer 2.

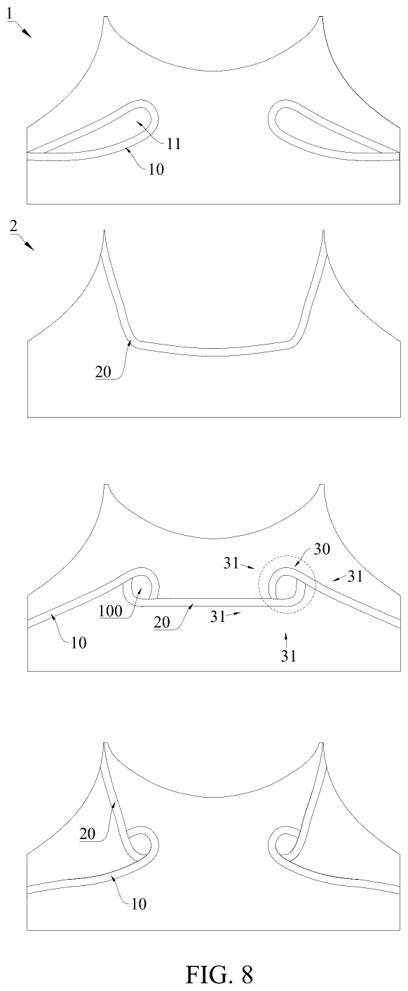

As shown in FIG. 8, a breast pump bra disclosed in an eighth embodiment of the present invention comprises a first fabric layer 1 and a second fabric layer 2, in which the first fabric layer 1 is provided with first notches 11 in two cups respectively from cup center areas 30 covering breasts to cup peripheral side areas 31 thereof, each of the first force-bearing edges 10 of each of the first notches 11 is closed downward concave. The overall shape of each of the second force-bearing edges 20 of the second fabric layer 2 is concave downwards, the second fabric layer 2 passes through the two first notches 11 and overlaps the first fabric layer 1 in a layered manner, two cross bearing openings 100 are formed at overlapping portions of second force-bearing edges 20 and first force-bearing edges 10 of the two first notches 11, under the action of an external force applied, the two cross bearing openings 100 are capable of enlarging opening areas thereof along the cup center areas 30 to the cup peripheral side areas 31; after the opening areas of the cross bearing openings 100 are enlarged after stretching, and after a flange port of a breast pump is placed, the cross bearing openings 100 are capable of surrounding and wrapping the outer diameter of a suction opening of the flange port of the breast pump by means of the tension of the first fabric layer 1 and the second fabric layer 2.

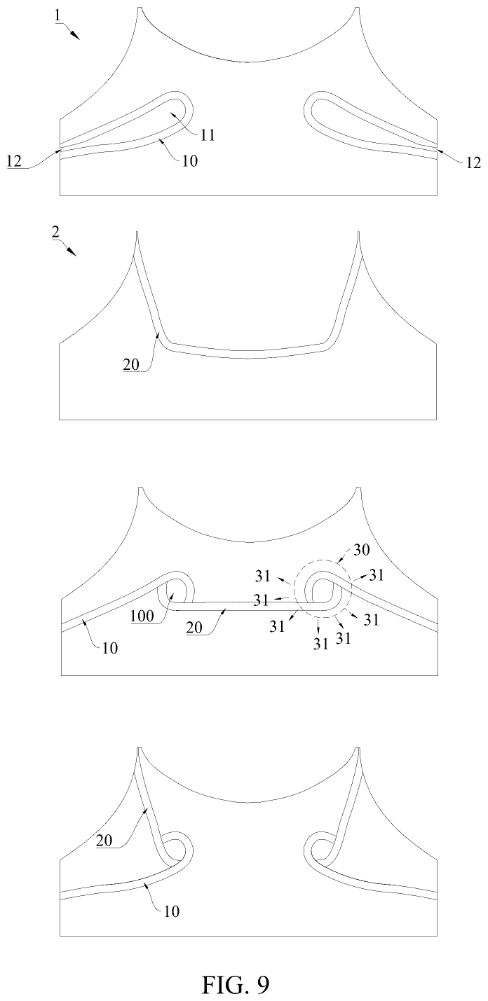

As shown in FIG. 9, a breast pump bra disclosed in a ninth embodiment of the present invention comprises a first fabric layer 1 and a second fabric layer 2, in which the first fabric layer 1 is provided with first notches 11 in two cups respectively from cup center areas 30 covering breasts to cup peripheral side areas 31 thereof, each of the first force-bearing edges 10 of each of the first notches 11 are concave downwards, each of the first notches 11 having a downward concave shape extends from each of the cup center areas 30 covering the breasts to an edge of each of the cup peripheral side areas 31 so as to form a first slit 12, the slits 12 enable the second fabric layer 2 to conveniently pass through the first notches 11.

The overall shape of each of the second force-bearing edges 20 of the second fabric layer 2 is concave downwards, the second fabric layer 2 passes through the two first notches 11 and overlaps the first fabric layer 1 in a layered manner, two cross bearing openings 100 are formed at overlapping portions of second force-bearing edges 20 and first force-bearing edges 10 of the two notches 11, under the action of an external force applied, the two cross bearing openings 100 are capable of enlarging opening areas thereof along the cup center areas 30 to the cup peripheral side areas 31, after a flange port of a breast pump is placed, the cross bearing openings 100 are capable of surrounding and wrapping the outer diameter of a suction opening of the flange port of the breast pump by means of the tension of the first fabric layer 1 and the second fabric layer 2.

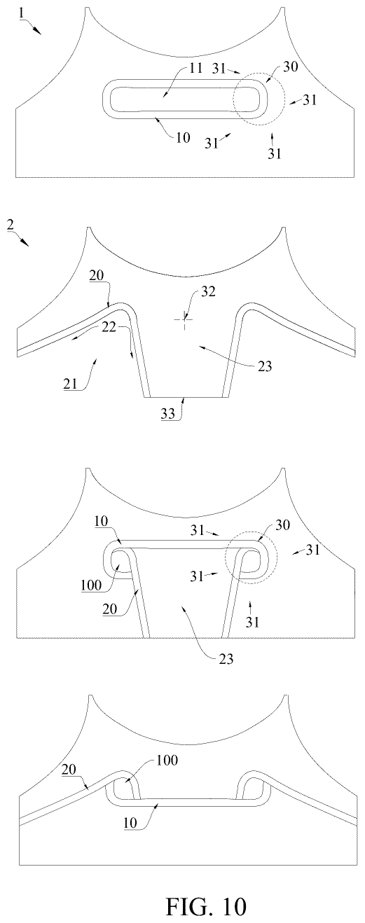

As shown in FIG. 10, a breast pump bra disclosed in a tenth embodiment of the present invention comprises a first fabric layer 1 and a second fabric layer 2, in which the first fabric layer 1 extends from the two cup center areas 30 of the two cups to a symmetric center 32 of the two cups in the cup peripheral side areas 31 thereof so as to form a first notch 11, so that the first force-bearing edge 10 of the first notch 11 forms a closed ring shape; and when an external force is applied to enlarge the first notch, the first force-bearing edge 10 of the first notch 11 limits the area of the first notch 11 to be enlarged under the elastic force thereof.

The second fabric layer 2 comprises two second notches 21, and the second force-bearing edges 20 of the two second notches 21 respectively extend from the two cup center areas 30 to edges of the cup peripheral side areas 31. Each of the second notches 21 having a downward concave shape extends from each of the cup center areas 30 covering the breasts to each of the cup peripheral side areas 31 in different directions to form a second slit 22, and the two slits 22 enable the second fabric layer 2 to extend downwards from the symmetry center 32 to a center front portion 33 so as to form a second lower portion 23 having a downward tension force. The lower portion 23 passes through the first notches 11 and overlaps the first fabric layer 1 in a layered manner, two cross bearing openings 100 are respectively formed at overlapping portions at the two sides of second force-bearing edges 20 and first force-bearing edges 10 of the two notches 11, under the action of an external force, the two cross bearing openings 100 are capable of enlarging opening areas thereof along the cup center areas 30 to the cup peripheral side areas 31, after a flange port of a breast pump is placed, the cross bearing openings 100 are capable of surrounding and wrapping the outer diameter of a suction opening of the flange port of the breast pump by means of the tension of the first fabric layer 1 and the second fabric layer 2.

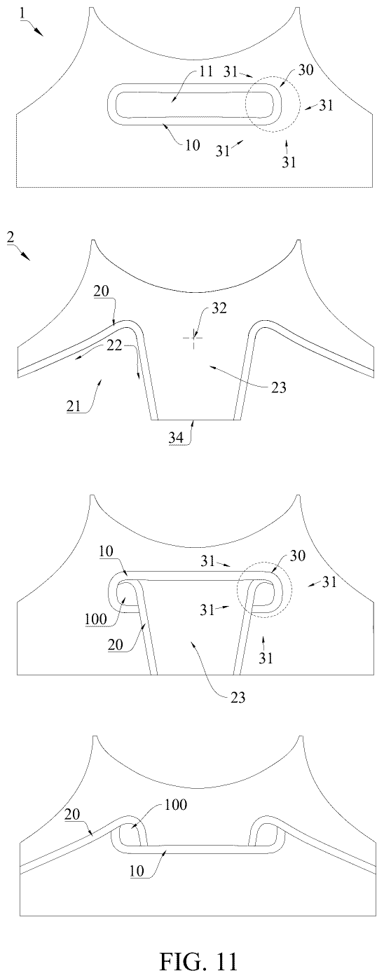

As shown in FIG. 11, a breast pump bra disclosed in an eleventh embodiment of the present invention comprises a first fabric layer 1 and a second fabric layer 2, in which the first fabric layer 1 extends from the two cup center areas 30 of the two cups to a symmetric center 32 of the two cups in the cup peripheral side areas 31 thereof so as to form a first notch 11, so that the first force-bearing edge 10 of the first notch 11 forms a closed ring shape; when the first notch is enlarged by an external force, the first force-bearing edge 10 of the first notch 11 limits the area of the first notch 11 to be enlarged under the elastic force thereof.

The second fabric layer 2 comprises two second notches 21 having an upward concave shape, and the second force-bearing edges 20 of the two second notches 21 respectively extend from the two cup center areas 30 to edges of the cup peripheral side areas 31. Each of the second notches 21 forms a second slit 22 extending to each of the cup peripheral side areas 31, the second fabric layer 2 passes through the first notches 11 via the second slits 22 and overlaps the first fabric layer 1 in a layered manner, two cross bearing openings 100 are respectively formed at overlapping portions at the two sides of second force-bearing edges 20 of the two second notches 21 and first force-bearing edges 10 of the two first notches 11, under the action of an external force, the two cross bearing openings 100 are capable of enlarging opening areas thereof along the cup center areas 30 to the cup peripheral side areas 31, after a flange port of a breast pump is placed, the cross bearing openings 100 are capable of surrounding and wrapping the outer diameter of a suction opening of the flange port of the breast pump by means of the tension of the first fabric layer 1 and the second fabric layer 2.

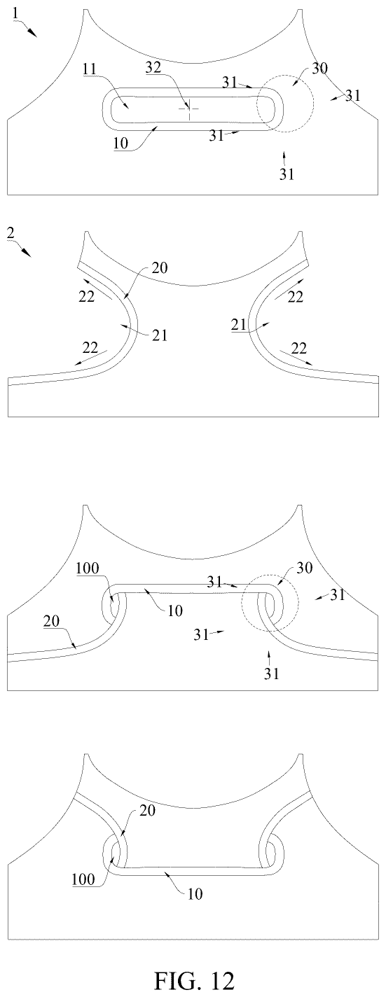

As shown in FIG. 12, a breast pump bra disclosed in a twelfth embodiment of the present invention comprises a first fabric layer 1 and a second fabric layer 2, in which the first fabric layer 1 extends from the two cup center areas 30 of the two cups to a symmetric center 32 of the two cups in the cup peripheral side areas 31 thereof so as to form a first notch 11, so that the first force-bearing edge 10 of the first notch 11 forms a closed ring shape; when the first notch is enlarged by an external force, the first force-bearing edge 10 of the first notch 11 has a tension force limiting the area of the first notch 11 to be enlarged.

Two second notches 21 on the light and right are cut at the second fabric layer 2 in two cups respectively from cup center areas 30 covering breasts to the edges of cup peripheral side areas 31 thereof, each of the second notches 21 forms a second slit 22 extending to each of the cup peripheral side areas 31, the second fabric layer 2 passes through each of the first notches 11 and overlaps with the first fabric layer 1 in a layered manner, two cross bearing openings 100 are formed at overlapping portions of second force-bearing edges 20 of the second notches 21 and first force-bearing edges 10 of the first notches 11, under the action of an external force, the two cross bearing openings 100 are capable of enlarging opening areas thereof along the cup center areas 30 to the cup peripheral side areas 31; after a flange port of a breast pump is placed, the cross bearing openings 100 are capable of surrounding and wrapping the outer diameter of a suction opening of the flange port of the breast pump by means of the tension of the first fabric layer 1 and the second fabric layer 2.

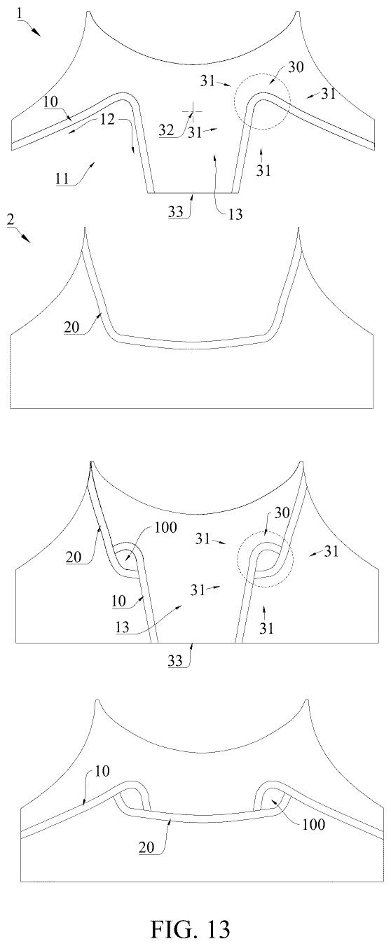

As shown in FIG. 13, a breast pump bra disclosed in a thirteenth embodiment of the present invention comprises a first fabric layer 1 and a second fabric layer 2, in which the first fabric layer 1 is provided with first notches 11 having a downward concave shape in two cups respectively from cup center areas 30 covering breasts to cup peripheral side areas 31 thereof, two first notches 11 having a downward concave shape are provided, and the two first notches 11 extend from the cup center areas 30 covering the breasts to the corresponding cup peripheral side areas 31 so as to form first slits 12, and the two first slits 12 enable the first fabric layer 1 to extend downwards from the symmetrical center 32 to the center front portion 33 so as to form a first lower portion 13 having a downward tension force,

the overall shape of each of the second force-bearing edges 20 of the second fabric layer 2 is concave downwards, the second fabric layer 2 passes through the two first notches 11 and overlaps the first fabric layer 1 in a layered manner, two cross bearing openings 100 are formed at overlapping portions of second force-bearing edges 20 and first force-bearing edges 10 of the two notches 11, under the action of an external force, the two cross bearing openings 100 are capable of enlarging opening areas thereof along the cup center areas 30 to the cup peripheral side areas 31, after a flange port of a breast pump is placed, the cross bearing openings 100 are capable of surrounding and wrapping the outer diameter of a suction opening of the flange port of the breast pump by means of the tension of the first fabric layer 1 and the second fabric layer 2.

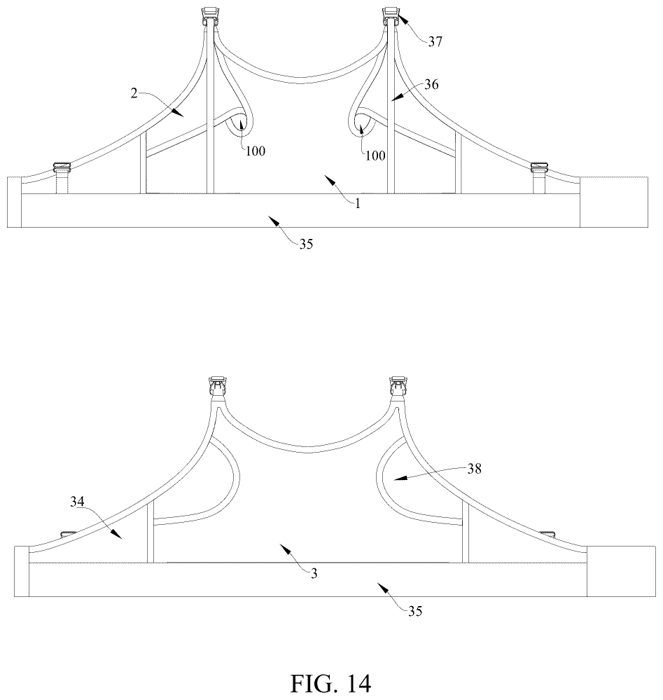

As shown in FIG. 14, a breast pump bra disclosed in a fourteenth embodiment of the present invention further comprises a third fabric layer 3, side panels 34 and an underband 35, in which the third fabric layer 3 covers an outer side of the first fabric layer 1 or the second fabric layer 2, and covers the cross bearing openings 100. The third fabric layer 3 is respectively provided with side notches 38 at the left and right sides, the side notches 38 facilitate finding an insertion opening, and the first fabric layer 1, the second fabric layer 2 and the third fabric layer 3 are fixedly connected to the side panels 34 and the underband 35 respectively.

From the above description, it can be seen that the above embodiment of the present invention achieves the following technical effects.

1. By setting the first notches on the first fabric layer, and enabling side parts at two sides of the second fabric layer to respectively pass through the two first notches and be stacked on the first fabric layer, the bearing openings capable of surrounding and wrapping a flange port of a breast pump is formed between an edge of each of the first notches and an edge of a side part of the second fabric layer, and the breast pump has more stable bearing.

2. The bearing openings formed by the cross-stacking of two fabrics can be suitable for breast pumps having various sizes.

3. The bearing openings formed by cross-stacking of two fabrics can reduce the limitation of the bra structure, making it more comfortable to wear compared to a breast pump bra having bearing openings formed by single piece overlapping.

The foregoing is merely a preferred embodiment of the present invention and is not intended to limit the present invention which may be subject to various modifications and variations to a person skilled in the art. Any modification, equivalent replacement, improvement, etc. made within the spirit and principles of the present invention should be included in the scope of protection of the present invention.

What is claimed is:

1. A breast pump bra, comprising a first fabric layer and a second fabric layer, wherein the first fabric layer is provided with first notches from cup center areas covering breasts to cup peripheral side areas, the second fabric layer passes through each of the first notches and overlaps the first fabric layer in a layered manner, cross bearing openings are formed at overlapping portions of first force-bearing edges of the first fabric layer and second force-bearing edges of the second fabric layer, under the action of an external force, each of the cross bearing openings is capable of enlarging an opening area thereof along each of the cup center areas to each of the cup peripheral side areas so as to place a flange port of a breast pump, and each of the cross bearing openings is capable of surrounding and wrapping the outer diameter of a suction opening of the flange port of the breast pump by means of tension of fabrics of the first fabric layer and the second fabric layer.

2. The breast pump bra according to claim 1,
wherein each of the first force-bearing edges of the first fabric layer and each of the second force-bearing edges of the second fabric layer are both concave, each of the first force-bearing edges is opposite to each of the second bearing edges in a recess direction at each of the cross bearing openings, and two tension directions for reducing the opening area of each of the cross bearing openings formed at the cross bearing openings by the second fabric layer and the first fabric layer are opposite.

3. The breast pump bra according to claim 2,
wherein each of the first notches extends from each of the cup center areas covering the breasts to an edge of each of the cup peripheral side areas so as to form a first slit, and each of the first slits enables the second fabric layer to conveniently pass through each of the first notches.

4. The breast pump bra according to claim 2,
wherein the first fabric layer comprises two first notches, and the two first notches respectively extend from the cup center areas to edges of the cup peripheral side areas.

5. The breast pump bra according to claim 2,
wherein the second fabric layer comprises two second notches, and the second force-bearing edges of the two second notches respectively extend from the two cup center areas to the edges of the cup peripheral side areas.

6. The breast pump bra according to claim 5,
wherein each of the second notches forms a second slit at the edge of each of the cup peripheral side areas.

7. The breast pump bra according to claim 1,
wherein the first notches are respectively formed by extending from the two cup center areas to a symmetric center of two cups in the cup peripheral side areas, so that the first force-bearing edge of each of the first notches forms a closed ring shape; and when an external force is applied to enlarge each of the first notches, the first force-bearing edge of each of the first notches has tension limiting the area of each of the first notches to be enlarged.

8. The breast pump bra according to claim 7,
wherein the second fabric layer comprises two second notches, and the second force-bearing edges of the two second notches respectively extend from the two cup center areas to the edges of the cup peripheral side areas.

9. The breast pump bra according to claim 8,
wherein each of the second notches forms a second slit at the edge of each of the cup peripheral side areas.

10. The breast pump bra according to claim 8,
wherein each of the second notches extends from each of the cup center areas covering the breasts to each of the cup peripheral side areas in different directions to form a second slit, and the two slits enable the second fabric layer to extend downwards from the symmetry center to a center front portion so as to form a second lower portion having a downward tension force.

11. The breast pump bra according to claim 1,
wherein two first notches having a downward concave shape are provided, the two first notches extend from the cup center areas covering the breasts to the corresponding cup peripheral side areas so as to form first slits, the two first slits enable the first fabric layer to extend downwards from the symmetrical center to the center front portion so as to form a first lower portion having a downward tension force, and the second fabric layer passes through the two first notches respectively and overlaps the first fabric layer in a layered manner so as to form the bearing openings.

12. The breast pump bra according to claim 1,
further comprising a third fabric layer, wherein the third fabric layer covers an outer side of the first fabric layer or the second fabric layer, and covers the cross bearing openings.

13. The breast pump bra according to claim 12,
further comprising side panels and an underband, wherein the first fabric layer, the second fabric layer and the third fabric layer are fixedly connected to the side panels and the underband respectively.
